(19) United States
(12) Patent Application Publication
(10) Pub. No.: US 2007/0219236 A1
(43) Pub. Date: Sep. 20, 2007

(54) Dialkoxy-Imidazopyridines Derivatives

(75) Inventor: Wilm Buhr, Konstanz (DE)

Correspondence Address: NATH & ASSOCIATES PLLC, 112 South West Street, Alexandria, VA, 22314, US

(73) Assignee: Atlanta Pharma AG, Konstanz (DE)

(21) Appl. No.: 11/578,844

(22) PCT Filed: Apr. 26, 2005
(86) PCT No.: PCT/EP05/51851

§ 371 Date: Oct. 19, 2006

(30) Foreign Application Priority Data
Apr. 28, 2004 (EP) 04010042.2

(51) Int. Cl. C07D 471/04; A61K 31/44; A61P 1/04

(52) U.S. Cl. 514/303; 546/118

(57) ABSTRACT

The invention relates to compounds of formula 1 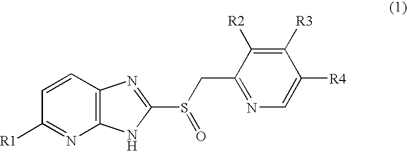 and to medicaments comprising these compounds.

### SUBJECT-MATTER OF THE INVENTION

The present invention relates to novel dialkoxy-imidazopyridines. The novel compounds can be used in the pharmaceutical industry for preparing medicaments.

### BACKGROUND OF THE INVENTION

Owing to their H⁺/K⁺-ATPase-inhibitory action, pyridin-2-ylmethylsulphinyl-1H-benzimidazoles, such as those known, for example, from EP-A-0005129, EP-A-0166287, EP-A-0174726, EP-A-0254588 and EP-A-0268956 are of considerable importance in the therapy of disorders associated with an increased secretion of gastric acid.

Examples of active compounds from this group which are commercially available or in clinical development are 5-methoxy-2-[(4-methoxy-3,5-dimethyl-2-pyridinyl)methylsulphinyl]-1H-benzimidazole (INN: omeprazole), (S)-5-methoxy-2-[(4-methoxy-3,5-dimethyl-2-pyridinyl)methylsulphinyl]-1H-benzimidazole (INN: esomeprazole), 5-difluoromethoxy-2-[(3,4-dimethoxy-2-pyridinyl)methylsulphinyl]-1H-benzimidazole (INN: pantoprazole), 2-[3-methyl-4-(2,2,2-trifluoroethoxy)-2-pyridinyl)methylsulphinyl]-1 H-benzimidazole (INN: lansoprazole), 2-{[4-(3-methoxypropoxy)-3-methylpyridin-2-yl]methylsulphinyl}-1H-benzimidazole (INN: rabeprazole) and 5-methoxy-2-((4-methoxy-3,5-dimethyl-2-pyridylmethyl)sulphinyl)-1H-imidazo[4,5-b]pyridine (INN: tenatoprazole).

The above mentioned sulphinyl derivatives are, owing to their mechanism of action, also referred to as proton pump inhibitors or, abbreviated, as PPI.

### DESCRIPTION OF THE RELATED ART

European patent application EP 187977 relates to tetrahydroquinoline and imidazopyridine derivatives and their use for the treatment of gastric and/or duodenal ulcers.

In European patent application EP 254588, a selection of certain imidazo[4,5-b]pyridine compounds of a general formula and their use for the treatment of gastric and/or duodenal ulcers is disclosed.

A common property of the abovementioned PPI is their sensitivity to acids (ultimately essential for effectiveness) which becomes apparent in their strong tendency to decompose in a neutral and in particular an acidic environment. The compounds disclosed in EP 254588, in particular the compound 5-methoxy-2-((4-methoxy-3,5-dimethyl-2-pyridylmethyl)sulphinyl)-1H-imidazo[4,5-b]pyridine (INN: tenatoprazole), are strong inhibitors of gastric acid secretion. However, these compounds are not very stable in neutral environment (at pH 7), which might elevate the risk of side effects, since the compounds are partly transformed in neutral or slightly acidic environment to highly reactive intermediates, which can react with enzymes and cells in the human body other than the H⁺/K⁺-ATPase located in the parietal cells of the stomach.

In addition to the European patent applications mentioned above, PPI with certain substitution pattern are also described in European patent applications EP 234690 and EP 533264.

### DESCRIPTION OF THE INVENTION

It has now been found that the compounds disclosed in more detail below show a strong inhibition of acid secretion and are simultaneously comparatively stable in neutral environment.

The invention relates to compounds of the general formula 1,

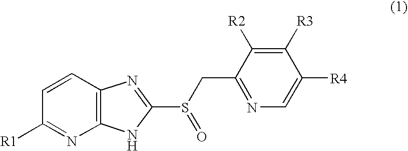

in which

R1 is 1-4C-alkoxy or 3-7C-cycloalkyl-1-4C-alkoxy,

R2 is 1-4C-alkoxy,

R3 is 1-4C-alkoxy or -4C-alkoxy-1-4C-alkoxy and

R4 is hydrogen or 1-4C-alkyl,

and the salts of these compounds.

1-4C-Alkyl represents straight-chain or branched alkyl groups having 1 to 4 carbon atoms. Examples which may be mentioned are the butyl, isobutyl, sec-butyl, tert-butyl, propyl, isopropyl, ethyl and, preferably, the methyl group.

1-4C-Alkoxy represents a group, which in addition to the oxygen atom contains one of the aforementioned 1-4C-alkyl groups. Examples which may be mentioned are the butoxy, isobutoxy, sec-butoxy, tert-butoxy, propoxy, isopropoxy, ethoxy and, preferably, the methoxy group.

3-7C-Cycloalkyl represents cyclopropyl, cyclobutyl, cyclopentyl, cyclohexyl and cycloheptyl, of which cyclopropyl, cyclobutyl and cyclopentyl are preferred.

3-7C-Cycloalkyl-1 -4C-alkoxy represents one of the aforementioned 1-4C-alkoxy groups, which is substituted by one of the aforementioned 3-7C-cycloalkyl groups. Examples which may be mentioned are the cyclohexylmethoxy, the cydohexylethoxy and, in particular, the cyclo-propylmethoxy group.

1-4C-Alkoxy-1-4C-alkoxy represents a 1-4C-alkoxy group, which is substituted by another 1-4C-alkoxy group. Examples which may be mentioned are the methoxy-ethoxy and the methoxy-propoxy group.

According to the invention, within the meaning of salts all salts with inorganic and organic bases are included, in particular the salts with alkali metals, such as the lithium, sodium and potassium salts, or the salts with alkaline earth metals, such as the magnesium and calcium salts, but also other pharmacologically compatible salts, such as, for example, the aluminium or the zinc salts. Particularly preferred are the sodium and the magnesium salts.

Pharmacologically incompatible salts, which can initially be obtained, for example, as process products in the production of the compounds according to the invention on the industrial scale, which are also within the scope of the invention, are—for the production of medicaments—converted into the pharmacologically tolerable salts by processes known to the person skilled in the art.

It is known to the person skilled in the art that the compounds according to invention and their salts, if, for example, they are isolated in crystalline form, can contain various amounts of solvents. The invention therefore also comprises all solvates and in particular all hydrates of the compounds of the formula 1, and also all solvates and in particular all hydrates of the salts of the compounds of the formula 1.

Preferred within the scope of the invention are compounds of the general formula 1, in which

R1 is methoxy or cyclopropylmethoxy,

R2 is methoxy,

R3 is methoxy, methoxy-ethoxy or methoxy-propoxy and

R4 is hydrogen or methyl,

and the salts of these compounds.

Particularly preferred within the scope of the invention are compounds of the general formula 1, in which

R1 is methoxy or cyclopropylmethoxy,

R2 is methoxy,

R3 is methoxy and

R4 is hydrogen or methyl,

and the salts of these compounds.

A particularly preferred compound within the scope of the invention is the compound 5-methoxy-2-[(3,4-dimethoxy-2-pyridylmethyl)sulphinyl]-1H-imidazo[4,5-b]pyridine and the hydrates of this compound, the salts of this compound and the hydrates of the salts of this compound.

Particularly preferred salts within the scope of the invention are the salts 5-methoxy-2-[(3,4-dimethoxy-2-pyridylmethyl)sulphinyl]-1H-imidazo[4,5-b]pyridine sodium and bis-5-methoxy-2-[(3,4-dimethoxy-2-pyridylmethyl)sulphinyl]-1H-imidazo[4,5-b]pyridine magnesium and the hydrates of these salts.

The compounds according to the invention are chiral compounds. The invention thus relates to the racemates as well as to the enantiomers and mixtures thereof in any desired ratio. In view of the fact that, from a medicinal point of view, it may be advantageous for certain chiral compounds to be administered in the form of the one or the other enantiomer, a preferred subject matter of the inventions are the enantiomers of the compounds of formula 1, preferably the enantiomers being substantially free of the respective other enantiomers with opposite configuration.

Accordingly, particularly preferred are on one hand the compounds with (S)-configuration of the general formula 1a

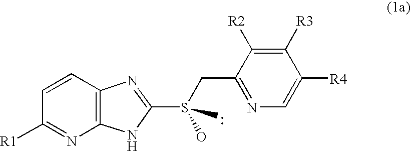

in which R1, R2, R3 and R4 have the meanings given above.

A particularly preferred compound with (S)-configuration within the scope of the invention is the compound (S)-5-methoxy-2-[(3,4-dimethoxy-2-pyridylmethyl)sulphinyl]-1H-imidazo[4,5-b]pyridine and the hydrates of this compound, the salts of this compound and the hydrates of the salts of this compound.

Particularly preferred salts of compounds with (S)-configuration are the salts (S)-5-methoxy-2-[(3,4-dimethoxy-2-pyridylmethyl)sulphinyl]-1H-imidazo[4,5-b]pyridine sodium and (S)-bis-5-methoxy-2-1(3,4-dimethoxy-2-pyridylmethyl)sulphinyl]-1H-imidazo[4,5-b]pyridine magnesium and the hydrates of these salts.

Particularly preferred are on the other hand the compounds with (R)-configuration of the general formula 1b

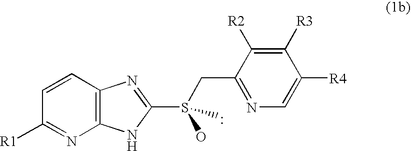

in which R1, R2, R3 and R4 have the meanings given above.

A particularly preferred compound with (R)-configuration within the scope of the invention is the compound (R)-5-methoxy-2-[(3,4-dimethoxy-2-pyridylmethyl)sulphinyl]-1H-imidazo[4,5-b]pyridine and the hydrates of this compound, the salts of this compound and the hydrates of the salts of this compound.

Particularly preferred salts of compounds with (R)-configuration are the salts (R)-5-methoxy-2-[(3,4-dimethoxy-2-pyridylmethyl)sulphinyl]-1H-imidazo[4,5-b]pyridine sodium and (R)-bis-5-methoxy-2-[(3,4-dimethoxy-2-pyridylmethyl)sulphinyl]-1H-imidazo[4,5-b]pyridine magnesium and the hydrates of these salts.

The compounds of formula 1, from which the compounds with (S)— and (R)-configuration (compounds of formula 1a and 1b) can be obtained, can be synthesized as described in European patent applications 166287 and 254588, and/or according to the following reaction scheme:

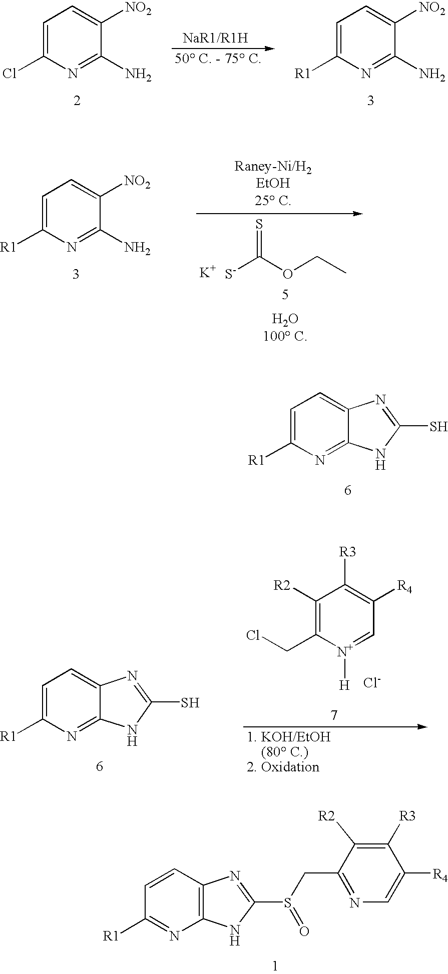

The separation of the compounds of formula 1 into the enantiomers can be accomplished according to various processes, for example as described in international patent application WO92/08716 or by column chromatography. Alternatively, the compounds of formulae 1a and 1b can be obtained by chiral oxidation of the sulphides (reaction product of compounds 6 and 7) as described in international patent applications WO96102535 (=U.S. Pat. No. 5,948,789), WO2004052882 or WO2004052881.

The salts of the compounds of formulae 1, 1a and 1b are prepared by processes known per se by reacting the compounds of formulae 1, 1a and 1b, which can be regarded as weak acids, with suitable bases, for example with alkali metal hydroxides or alkoxides, such as sodium hydroxide or sodium methoxide, or with alkaline earth metal alkoxides, such as magnesium methoxide. As an example, the magnesium salts of the compounds of formulae 1, 1a and 1b, which are—besides the sodium salts—the preferred salts, are prepared in a manner known per se by reacting compounds of formulae 1, 1a and 1b with a magnesium base, for example a magnesium alkoxide, or from a readily soluble salt of a compound of formulae 1, 1a or 1b (for example of a sodium salt) using a magnesium salt in water or in mixtures of water with polar organic solvents (for example alcohols, preferably methanol, ethanol or isopropanol, or ketones, preferably acetone).

Magnesium salts suitable for use in the process are, for example, magnesium chloride, magnesium bromide, magnesium fluoride, magnesium iodide, magnesium formate, magnesium acetate, magnesium propionate, magnesium gluconate or magnesium carbonate. It is also possible to react magnesium alkoxides (for example magnesium methoxide, magnesium ethoxide, magnesium (iso)propoxide, magnesium butoxide, magnesium hexoxide or magnesium phenoxide) in an alkoholate medium with the compounds of formulae 1, 1a and 1b or with a sodium salt thereof, and to crystallise the magnesium salt hydrates of the compounds of formulae 1, 1a and 1b by addition of water. Furthermore, it is possible to recrystallise obtained magnesium salt hydrates from, e.g., methanol/water mixtures.

According to the invention, “compounds with (S)-configuration” is understood to include “compounds with (S)-configuration being substantially free of compounds with (R)-configuraton”.

“Substantially free” in the context of the invention means that the compounds with (S)-configuration and/or their salts contain less than 10% by weight of compounds with (R)-configuration and/or their salts. Preferably, “substantially free” means that compounds with (S)-configuration and/or their salts contain less than 5% by weight of compounds with (R)-configuration and/or their salts. In the most preferred embodiment, “substantially free” means that compounds with (S)-configuration and/or their salts contain less than 1% by weight of compounds with (R)-configuration and/or their salts.

According to the invention, “compounds with (R)-configuration” is understood to include “compounds with (R)-configuration being substantially free of compounds with (S)-configuration”.

“Substantially free” in the context of the invention means that the compounds with (R)-configuration and/or their salts contain less than 10% by weight of compounds with (S)-configuration and/or their salts. Preferably, “substantially free” means that compounds with (R)-configuration and/or their salts contain less than 5% by weight of compounds with (S)-configuraton and/or their salts. In the most preferred embodiment, “substantially free” means that compounds with (R)-configuration and/or their salts contain less than 1% by weight of compounds with (S)-configuration and/or their salts.

The following examples serve to illustrate the invention in greater detail without restricting it. Likewise, further compounds of the formulae 1, 1a and 1b, the preparation of which is not described explicitly, can be prepared in an analogous manner or in a manner familiar per se to the person skilled in the art using customary process techniques. The abbreviation min stands for minute(s), h for hour(s). The novel compounds named expressly as examples, and any salts of these compounds, are preferred subject matter of the invention. Additional subject matter of the invention are compounds of formula 2

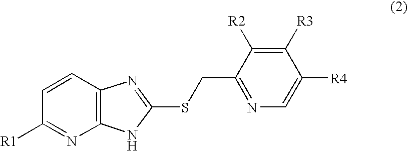

in which R1, R2, R3 and R4 have the meanings given above, and their salts, such as the hydrochloride, the sulfate, the phosphate or other salts with acids.

### EXAMPLES

Starting Compounds and Intermediates

### 2-(3,4-Dimethoxy-pyridine-2-ylmethylsulfanyl)-5-methoxy-3H-imidazo[4,5-b]pyridine

A reaction mixture of 10.00 g (55.20 mmol) 5-methoxy-3H4midazo[4,5-b]pyridine-2-thiol and 12.37 g (55.20 mmol) 2-chloromethyl-3,4-dimethoxy pyridinium chloride in isopropanol (200 ml) is stirred for 2 h under reflux. The mixture is concentrated, filtered and dried at 60° C. for 16 h. Afterwards the crude hydrochloride of the product is suspended in a mixture of water dichloromethane and is basified to pH 8 by adding sodium hydroxide solution (6 N). The mixture is extracted with dichloromethane three times. The combined organic layers are washed with water, dried over calcium chloride, concentrated in vacuo, reslurried in acetone and dried again in vacuo to give 14.76 g (44.4 mmol/80%) of the title product with a melting point of 157.0° C. (acetone).

Final Products of Formulae 1, 1a and 1b

### 1. 2-(3,4-Dimethoxy-pyridine-2-ylmethanesulfinyl)-5-methoxy-3H-imidazo[4,5-b]pyridine

To a at −10° C. cooled suspension of 12.35 g (37.15 mmol) 2-(3,4-dimethoxy-pyridine-2-ylmethylsulfanyl)-5-methoxy-3H-imidazo[4,5-b]pyridine in dichloromethane is added 11.00 g (˜50.00 mmol) 3-chloroperoxybenzoic acid (˜77%) dissolved in dichloromethane (110 ml) and the mixture is stirred for 1 h at 0° C. Subsequently the reaction is quenched by adding saturated sodium thiosulphate solution and sodium hydrogen carbonate solution. The mixture is extracted with dichloromethane three times. The combined organic layers are concentrated in vacuo and purified by column chromatography (dichloromethane/methanol: 100/1 to 13/1). This product is reslurried from acetone and dried in vacuo to give 8.12 g (23.3 mmol /63%) of the title product as a colourless solid.

¹H-NMR (200 MHz, D₆-DMSO):δ=3.78 (s, 3 H), 3.90 (s, 3 H), 3.92 (s, 3 H), 4.69 (d, 1H), 4.75 (d, 1 H), 6.80 (d, 1 H), 7.10 (d, 1 H), 7.99 (d, 1H), 8.14 (d, 1 H).

### 2. (S)-2-(3,4-Dimethoxy-pyridine-2-ylmethanesulfinyl)-5-methoxy-3H-imidazo[4,5-b]pyridine

6.00 g (17.22 mmol) of 2-(3,4-dimethoxy-pyridine-2-ylmethanesulfinyl)-5-methoxy-3H-imidazo[4,5-b]pyridine are separated by using chiral column chromatography (column:CHIRALPAK® AS-H 5 μm/mobile phase: 80/20 CO₂/MeOH+1% DEA/flow rate: 60 ml/min/outlet pressure: 150 bar/retention time: 13.5 min) to give 2.74 g (7.86 mmol/46%) of the title product.

[α]_{D}²⁰=−140° (c 0.005, chloroform/methanol: 1/1).

¹H-NMR (200 MHz, D₆-DMSO): δ=3.78 (s, 3 H), 3.90 (s, 3 H), 3.92 (s, 3 H), 4.69 (d, 1 H), 4.75 (d, 1 H), 6.80 (d, 1 H), 7.10 (d, 1 H), 7.99 (d, 1 H), 8.14 (d, 1 H).

### 3. (R)-2-(3,4-Dimethoxy-pyridine-2-ylmethanesulfinyl)-5-methoxy-3H-imidazo[4,5-b]pyridine

6.00 g (17.22 mmol) of 2-(3,4-dimethoxy-pyridine-2-ylmethanesulfinyl)-5-methoxy-3H-imidazo[4,5-b]pyridine are separated by using chiral column chromatography (column:CHIRALPAK® AS-H 5 μm/mobile phase: 80/20 CO₂/MeOH+1% DEA/flow rate: 60 ml/min/outlet pressure: 150 bar./retention time: 12.1 min) to give 2.86 g (8.21 mmol/48%) of the title product.

[α]_{D}²⁰=+130 retention time: 13.5 min ° (c 0.005, chloroform/methanol: 1/1).

¹H-NMR (200MHz, D₆-DMSO): δ=3.78 (s, 3 H), 3.90 (s, 3 H), 3.92 (s, 3 H), 4.69 (d, 1 H), 4.75 (d, 1 H), 6.80 (d, 1 H), 7.10 (d, 1 H), 7.99 (d, 1 H), 8.14 (d, 1 H).

Commercial Utility

The compounds of the general formula 1 and their salts and hydrates, and the hydrates of the salts (hereinafter “compounds of the invention”) have useful pharmacological properties, rendering them commercially utilizable. In particular, they have a pronounced inhibitory effect on the secretion of gastric acid and excellent gastrointestinal protective action in warm-blooded animals, in particular man. Here, the compounds according to the invention are distinguished by a highly selective action, an advantageous duration of action, a particularly high bioavailability, a metabolization profile that is uniform among different individuals, the lack of significant side-effects and a wide therapeutic spectrum.

In this context, “gastrointestinal protection” is to be understood as the prevention and treatment of gastrointestinal disorders, in particular gastrointestinal inflammatory disorders and lesions (such as, for example, Ulcus ventriculi, Ulcus duodeni, gastritis, irritable bowel owing to an increased production of acid or as a result of medicaments, GERD, Crohn's disease, IBD) which may be caused, for example, by microorganisms (for example Helicobacter pylori), bacterial toxins, medicaments (for example certain antiphlogistics and antirheumatic drugs), chemicals (for example ethanol), gastric acid or stress.

With their excellent properties, the compounds according to the invention, in various models for the determination of antiulcerogenic and antisecretory properties, surprisingly prove to be clearly superior to the prior art compounds, in particular with respect to their stability and their metabolization properties. Owing to these properties, the compounds according to the invention are highly suitable for use in human and veterinary medicine, where they are used, in particular, for the treatment and/or prophylaxis of gastrointestinal disorders.

Accordingly, the invention furthermore provides the use of the compounds according to the invention for the treatment and/or prophylaxis of the abovementioned diseases.

The invention also embraces the use of the compounds according to the invention for preparing medicaments used for the treatment and/or prophylaxis of the abovemenfioned diseases.

The invention also provides medicaments comprising the compounds according to the invention. In particular, the invention provides medicaments for oral use in solid form, containing the compounds of formula 1 in the form of their salts, in particular in the form of a sodium or magnesium salt, and/or in the form of a hydrate of such salt.

The medicaments are prepared by processes known per se which are familiar to the person skilled in the art. As medicaments, the compounds according to the invention are employed either as such or, preferably, in combination with suitable pharmaceutical auxiliaries or carriers in the form of tablets, coated tablets, capsules, suppositories, plasters (for example as TTS), emulsions, suspensions or solutions, where the content of active compound is advantageously from about 0.1 to about 95% and where it is possible to produce pharmaceutical dosage forms (for example flow-release forms or enteric forms) which, by the appropriate choice of auxiliaries and carriers, are tailored for the active compound and/or the desired onset of action and/or the duration of action.

The auxiliaries or carriers suitable for the desired pharmaceutical formulations are known to the person skilled in the art. In addition to solvents, gel formers, suppository bases, tabletting auxiliaries and other carriers for active compounds, it is possible to use, for example, antioxidants, dispersants, emulsifiers, antifoams, flavour-masking agents, preservatives, solubilizers, colorants or, in particular, permeation promoters and complex formers (for example cyclodextrins).

The compounds according to the invention can be administered orally, parenterally or percutaneously.

In human medicine, it has generally been found to be advantageous to administer the compounds according to the invention, when given orally, in a daily dose of from about 0.1 to about 2, preferably about 0.2 to about 1.5 and in particular about 0.3 to about 1.1, mg/kg of body weight [calculated on the basis of the compounds according to the invention in free form, i.e. not in salt form (=“free compound”], if appropriate in the form of a plurality of, preferably 1 to 4, individual doses, to obtain the desired result. For parenteral treatment, it is possible to use similar or (in particular when the active compounds are administered intravenously) generally lower dosages. The optimum dosage and the type of administration of the active compounds required in each case can easily be determined by the person skilled in the art.

A further aspect of the invention is thus a medicament, comprising a compound according to the invention together with customary auxiliaries, where the single dose comprises from about 10 to about 100 mg of the free compound.

A further aspect of the invention is a medicament, comprising a compound according to the invention together with customary auxiliaries, where the single dose comprises from about 20 to about 80 mg of the free compound.

A further aspect of the invention is the use of the compounds according to the invention for treating gastrointestinal disorders.

A further aspect of the invention is the use of the compounds according to the invention for treating gastrointestinal disorders in patients who are slow metabolizers.

A further aspect of the invention is the use of the compounds according to the invention hereof for treating gastrointestinal disorders in patients who have a risk of drug interactions.

A further aspect of the invention is the use of the compounds according to the invention for treating gastrointestinal disorders in patients who need an inhibition of acid secretion for an extended period of time.

A further aspect of the invention is a medicament for treating gastrointestinal disorders for use in patients who are slow metabolizers, comprising a compound according to the invention together with customary auxiliaries, where the single dose comprises from about 10 to about 100 mg of free compound

A further aspect of the invention is a medicament for treating gastrointestinal disorders for use in patients who are slow metabolizers, comprising a compound according to the invention together with customary auxiliaries, where the single dose comprises from about 20 to about 80 mg of free compound.

A further aspect of the invention is a medicament for treating gastrointestinal disorders for use in patients who have a risk of drug interactions, comprising a compound according to the invention together with customary auxiliaries, where the single dose comprises from about 10 to about 100 mg of free compound.

A further aspect of the invention is a medicament for treating gastrointestinal disorders for use in patients who have a risk of drug interactions, comprising a compound according to the invention together with customary auxiliaries, where the single dose comprises from about 20 to about 80 mg of free compound.

A further aspect of the invention is a medicament for treating gastrointestinal disorders for use in patients who need an inhibition of acid secretion for an extended period of time, comprising a compound according to the invention together with customary auxiliaries, where the single dose comprises from about 10 to about 100 mg of free compound.

A further aspect of the invention is a medicament for treating gastrointestinal disorders for use in patients who need an inhibition of acid secretion for an extended period of time, comprising a compound according to the invention together with customary auxiliaries, where the single dose comprises from about 20 to about 80 mg of free compound.

A further aspect of the invention is a medicament for treating gastrointestinal disorders for use in patients who are slow metabolizers, comprising in an oral solid application form a salt according to the invention or a hydrate thereof together with customary auxiliaries, where the single dose comprises from about 10 to about 100 mg of free compound.

A further aspect of the invention is a medicament for treating gastrointestinal disorders for use in patients who are slow metabolizers, comprising in an oral solid application form a salt according to the invention or a hydrate thereof together with customary auxiliaries, where the single dose comprises from about 20 to about 80 mg of free compound.

A further aspect of the invention is a medicament for treating gastrointestinal disorders for use in patients who have a risk for drug interactions, comprising in an oral solid application form a salt according to the invention or a hydrate thereof together with customary auxiliaries, where the single dose comprises from about 10 to about 100 mg of free compound.

A further aspect of the invention is a medicament for treating gastrointestinal disorders for use in patients who have a risk for drug interactions, comprising in an oral solid application form a salt according to the invention or a hydrate thereof together with customary auxiliaries, where the single dose comprises from about 20 to about 80 mg of free compound.

A further aspect of the invention is a medicament for treating gastrointestinal disorders for use in patients who need an inhibition of acid secretion for an extended period of time, comprising in an oral solid application form a salt according to the invention or a hydrate thereof together with customary auxiliaries, where the single dose comprises from about 10 to about 100 mg of free compound.

A further aspect of the invention is a medicament for treating gastrointestinal disorders for use in patients who need an inhibition of acid secretion for an extended period of time, comprising in an oral solid application form a salt according to the invention or a hydrate thereof together with customary auxiliaries, where the single dose comprises from about 20 to about 80 mg of free compound.

If the compounds according to the invention are to be used for treating the abovementioned diseases, the pharmaceutical preparations may also comprise one or more pharmacologically active ingredients from other groups of medicaments. Examples that may be mentioned include tranquilizers (for example from the group of the benzodiazepines, e.g., diazepam), spasmolytic drugs (e.g., bietamiverine or camylofine), anticholinergic drugs (e.g., oxyphencyclimine or phencarbamide), local anesthetics (e.g., tetracaine or procaine), and optionally also enzymes, vitamins or amino acids.

In this context, particular emphasis is given to the combination of the compounds according to the invention with other pharmaceuticals which buffer or neutralize gastric acid or which inhibit the secretion of acid, such as, for example, antacids (such as, for example, magaldrate) or H₂ blockers (e.g., cimetidine, ranitidine), and with gastrin antagonists with the aim to enhance the main action in an additive or superadditive sense and/or to eliminate or reduce side-effects or to obtain a more rapid onset of action. Mention may also be made of the fixed or free combination with NSAIDs (such as, for example, etofenamate, diclofenac, indometacin, ibuprofen or piroxicam) for preventing the gastrointestinal damage caused by the NSAIDs, or with compounds, which modify gastrointestinal motility, or with compounds, which reduce the incidence of transient lower esophageal sphincter relaxation (TLOSR), or with antibacterial substances (such as, for example, cephalosporins, tetracyclins, penicillins, macrolides, nitroimidazoles or else bismuth salt) for controlling Helicobacter pylori. Antibacterial combination partners that may be mentioned include, for example, meziocillin, ampicillin, amoxicillin, cefalothin, cefoxitin, cefotaxim, imipenem, gentamycin, amicacin, erythromycin, ciprofloxacin, metronidazole, clarithromycin, azithromycin and combinations thereof (e.g., clarithromycin+metronidazole or amoxicillin+clarithromycin).

Pharmacology

The excellent gastric protective action and the gastric acid secretion-inhibiting action of the compounds according to the invention can be demonstrated in investigations on animal experimental models. The compounds according to the invention investigated in the model mentioned below have been provided with numbers which correspond to the numbers of these compounds in the examples.

Testing of the Secretion-Inhibiting Action on the Perfused Rat Stomach

In Table A which follows, the influence of the compounds according to the invention on the pentagastrin-stimulated acid secretion of the perfused rat stomach after intraduodenal administration in vivo is shown.

TABLE A

| | Dose | Inhibition of |
|---|---|---|
| Example | (μmol/kg) | acid secretion |
| No. | i.d. | (%) |
| 1 | 2 | >50 |
| 2 | 2 | >50 |
| 3 | 2 | >50 |

Methodology

The abdomen of anesthetized rats (CD rat, female, 200-250 g; 1.5 g/kg i.m. urethane) was opened after tracheotomy by a median upper abdominal incision and a PVC catheter was fixed transorally in the esophagus and another via the pylorus such that the ends of the tubes just projected into the gastric lumen. The catheter leading from the pylorus led outward into the right abdominal wall through a side opening.

After thorough rinsing (about 50-100 ml), warm (37° C.) physiological NaCl solution was continuously passed through the stomach (0.5 ml/min, pH 6.8-6.9; Braun-Unita I). The pH (pH meter 632, glass electrode EA 147; Φ=5 mm, Metrohm) and, by titration with a freshly prepared 0.01N NaOH solution to pH 7 (Dosimat 665 Metrohm), the secreted HCl were determined in the effluent in each case collected at an interval of 15 minutes.

The gastric secretion was stimulated by continuous infusion of 1 μg/kg (=1.65 ml/h) of i.v. pentagastrin (left femoral vein) about 30 min after the end of the operation (i.e. after determination of 2 preliminary fractions). The substances to be tested were administered intraduodenally in a 2.5 ml/kg liquid volume 60 min after the start of the continuous pentagastrin infusion.

The body temperature of the animals was kept at a constant 37.8-38° C. by infrared irradiation and heat pads (automatic, stepless control by means of a rectal temperature sensor).

1. A compound of the general formula 1, 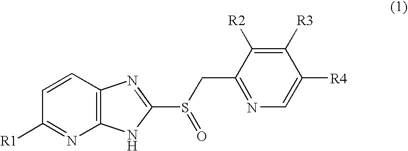
in which
R1 is 1-4C-alkoxy or 3-7C-cycloalkyl-1-4C-alkoxy,
R2 is 1-4C-alkoxy,
R3 is 1-4C-alkoxy or 1-4C-alkoxy-1-4C-alkoxy and
R4 is hydrogen or 1-4C-alkyl,
or a hydrate, salt or hydrate of a salt thereof

2. A compound of the general formula 1 according to claim 1, in which
R1 is methoxy or cyclopropylmethoxy,
R2 is methoxy,
R3 is methoxy, methoxy-ethoxy or methoxy-propoxy and
R4 is hydrogen or methyl,
or a hydrate, salt or hydrate of a salt thereof.

3. A compound of the general formula 1 according to claim 1, in which
R1 is methoxy or cyclopropylmethoxy,
R2 is methoxy,
R3 is methoxy and
R4 is hydrogen or methyl,
or a hydrate, salt or hydrate of a salt thereof.

4. Compound A compound according to claim 1, which is 5-methoxy-2-[(3,4-dimethoxy-2-pyridylmethyl)sulphinyl]-1H-imidazo[4,5-b]pyridine or a hydrate, salt or hydrate of a salt thereof.

5. A compound according to claim 1 with (S)-configuration, characterized by the general formula 1a, 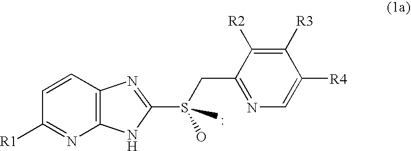
in which
R1 is 1-4C-alkoxy or 3-7C-cycloalkyl-1-4C-alkoxy,
R2 is 1-4C-alkoxy,
R3 is 1-4C-alkoxy or 1-4C-alkoxy-1-4C-alkoxy and
R4 is hydrogen or 1-4C-alkyl,
or a hydrate, salt or hydrate of a salt thereof.

6. Compound A compound according to claim 5, which is (S)-5-methoxy-2-[(3,4-dimethoxy-2-pyridylmethyl)sulphinyl]-1H-imidazo[4,5-b]pyridine or a hydrate, salt or hydrate of a salt thereof.

7. A salt of a compound according to claim 5, which is selected from the group consisting of (S)-5-methoxy-2-[(3,4-dimethoxy-2-pyridylmethyl)sulphinyl]-1H-imidazo[4,5-b]pyridine sodium, (S)-bis-5-methoxy-2-[(3,4-dimethoxy-2-pyridylmethyl)sulphinyl]-1H-imidazo[4,5-b]pyridine magnesium, and the hydrates thereof.

8. A compound according to claim 1 with (R)-configuration, characterized by the general formula 1b, 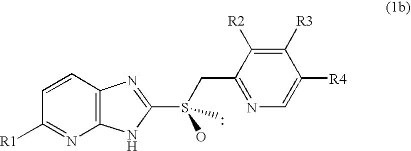
in which
R1 is 1-4C-alkoxy or 3-7C-cycloalkyl-1-4C-alkoxy,
R2 is 1-4C-alkoxy,
R3 is 1-4C-alkoxy or 1-4C-alkoxy-1-4C-alkoxy and
R4 is hydrogen or 1-4C-alkyl,
or a hydrate, salt or hydrate of a salt thereof.

9. A compound according to claim 8, which is (R)-5-methoxy-2-[(3,4-dimethoxy-2-pyridylmethyl)sulphinyl]-1H-imidazo[4,5-b]pyridine or a hydrate, salt or hydrate of a salt thereof.

10. A salt of a compound according to claim 8, which is selected from the group consisting of (R)-5-methoxy-2-[(3,4-dimethoxy-2-pyridylmethyl)sulphinyl]-1H-imidazo[4,5-b]pyridine sodium, (R)-bis-5-methoxy-2-[(3,4-dimethoxy-2-pyridylmethyl)sulphinyl]-1H-imidazo[4,5-b]pyridine magnesium, and the hydrates thereof.

11. A pharmaceutical composition comprising a compound according to claim 1, or a hydrate, salt or hydrate of a salt thereof, together with a pharmaceutically acceptable auxiliary.

12. A pharmaceutical composition comprising a compound according to claim 1, or a hydrate, salt or hydrate of a salt thereof, together with a pharmaceutically acceptable auxiliary, wherein a single dose comprises from about 10 to about 100 mg of the compound of formula 1.

13. (canceled)

14. A compound of formula 2 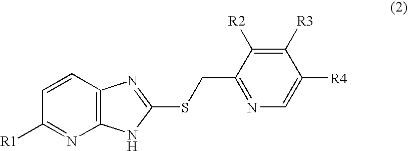
in which R1, R2, R3 and R4 have the meanings given in claim 1, or a hydrate, salt or hydrate of a salt thereof.

15. A method of treating a gastrointestinal disorder in a patient comprising administering to a patient in need thereof a therapeutically effective amount of a compound of formula 1 according to claim 1, or a hydrate, salt or hydrate of a salt thereof.
